(19) United States
(12) Patent Application Publication
(10) Pub. No.: US 2003/0158416 A1
(43) Pub. Date: Aug. 21, 2003

(54) Process for producing paroxetine salts substantially free from organic solvents

(75) Inventors: Shu-Zhong Wang, Yokohama-shi (JP); Erika Saito, Chiba-shi (JP); Naoya Shimokawa, Ischihara-shi (JP)

Correspondence Address: OBLON, SPIVAK, MCCLELLAND, MAIER & NEUSTADT, P.C., 1940 DUKE STREET, ALEXANDRIA, VA, 22314, US

(73) Assignee: ASAHI GLASS COMPANY LIMITED, Chiyoda-ku (JP)

(21) Appl. No.: 10/387,394

(22) Filed: Mar. 14, 2003

Related U.S. Application Data
(63) Continuation of application No. PCT/JP01/07967, filed on Sep. 13, 2001.

(30) Foreign Application Priority Data
Sep. 14, 2000 (JP) 2000-280037

(51) Int. Cl. C 07D 4 7/02

(52) U.S. Cl. 546/197

(57) ABSTRACT

A paroxetine salt free from an organic solvent is produced by substantially and safely removing the organic solvents from paroxetine hydrochloride.

A process for producing a paroxetine salt, which comprises neutralizing paroxetine hydrochloride containing an organic solvent into paroxetine, then forming a salt of paroxetine with an acid other than hydrochloric acid, and crystallizing the salt from an organic solvent.

### TECHNICAL FIELD

The present invention relates to a process for producing paroxetine salts, which have an inhibitory action on 5-hydroxytryptamine (5-HT) and are useful as therapeutic agents for various diseases such as depression and Parkinson's disease.

### BACKGROUND ART

Production of paroxetine salts, i.e., salts of (3S,4R)-3-[5-(1,3-dioxaindanyl)oxymethyl]-4-(p-fluorophenyl)piperidine, as medicines involves reactions using organic solvents, and therefore, paroxetine salts are often produced with organic solvents in them. However, in view of the use of paroxetine salts as medicines, removal of organic solvents to a medicinally unproblematic level is necessary to provide paroxetine salts substantially free from organic solvents.

For removal of organic solvent from paroxetine salts, (1) vacuum drying of paroxetine hydrochloride containing an organic solvent with gradual changes in drying conditions and, optionally, irradiation with microwaves (WO00/8017) and (2) replacement of an organic solvent in paroxetine hydrochloride containing the organic solvent with water followed by dehydration (WO96/24595) have been proposed.

In method (1), because of the necessity to change the drying conditions in accordance with the organic solvent content, the difficult control of the drying operation is problematic. Besides, it is difficult to completely remove an organic solvent by this method, and the organic solvent content is likely to go down beyond 1 mass %. Vacuum drying under heating at 60-70° C. for as much time as 13 hours is necessary to lower the organic solvent content to less than 1 mass %.

Method (2) is not favorable in view of quality control of medicines because of the possibility of contamination by the crystalline hydrate of paroxetine hydrochloride. For example, WO95/16448 discloses that unfavorable pink tablets are obtained when water remains in paroxetine salts as a consequence of incomplete dehydration in the production process. This method also has the problem that it is difficult to lower the solvent content to less than 0.1 mass %.

The present invention provides a process for producing a paroxetine salt other than the hydrochloride which is substantially free from an organic solvent by removing the organic solvents in paroxetine hydrochloride efficiently and absolutely. The present invention provides crystalline paroxetine acetate substantially free from an organic solvent.

### DISCLOSURE OF THE INVENTION

The present invention provides a process for producing a paroxetine salt substantially free from an organic solvent, which comprises neutralizing paroxetine hydrochloride containing an organic solvent into paroxetine, then forming a salt of paroxetine with an acid other than hydrochloric acid, and crystallizing the salt from an organic solvent to give a salt other than paroxetine hydrochloride.

### BEST MODE FOR CARRYING OUT THE INVENTION

In the present invention, the paroxetine hydrochloride containing an organic solvent is paroxetine hydrochloride containing a certain kind of organic solvent in a certain proportion. The paroxetine hydrochloride containing an organic solvent in the present invention may contain one kind of organic solvent or two or more kinds of organic solvents.

The organic solvent in the paroxetine hydrochloride containing an organic solvent (hereinafter referred to as organic solvent (X)) may, for example, be a hydrocarbon (such as pentane, hexane, heptane, octane, nonane, decane, undecane or dodecane), an alcohol (such as methanol, ethanol, 1-propanol, 2-propanol, 1-butanol, isobutanol, t-butanol or ethylene glycol), a ketone (such as acetone or 2-butanone), an ester (such as ethyl acetate), an ether (such as diethyl ether, diphenyl ether, tetrahydrofuran, 1,2-dimethoxyethane or 1,4-dioxane), a chlorohydrocarbon (such as chloroform, dichloromethane, carbon tetrachloride, 1,2-dichloroethane, chlorobenzene or dichlorobenzene), an aromatic hydrocarbon (such as benzene, toluene or xylene), an aromatic heterocycle (such as pyridine, quinoline or 2,6-dimethylpyridine), an amide (such as N,N-dimethylformamide, N,Ndimethylacetamide, N-methylpyrrolidinone) or other organic solvents (such as acetonitrile, dimethyl sulfoxide, 1,3-dimethyl-2-imidazolidinone, hexamethylphosphoramide or acetic acid).

In the process of the present invention, it is possible to remove an organic solvent (X) which is difficult to remove by ordinary drying techniques or an organic solvent (X) from paroxetine hydrochloride containing too small an amount of an organic solvent (X) to remove by ordinary drying techniques.

Organic solvents(X) which are difficult to remove by ordinary drying techniques include 1-butanol, 2-propanol, 1-propanol, ethanol, pyridine, acetonitrile, acetone, tetrahydrofuran, chloroform, toluene, acetic acid, ethyl acetate, N,N-dimethylformamide, quinoline, ethylene glycol, 1,4-dioxane, dimethyl sulfoxide, 1,3-dimethyl-2-imidazolidinone, hexamethylphosphoramide, 2,6-dimethylpyridine, xylene, chlorobenzene, t-butanol, diphenyl ether and the like.

Too small an amount of an organic solvent (X) to remove by ordinary techniques preferably means from more than 0.1 to 20 mass % (in particular from 1 to 10 mass %) of the organic solvent in paroxetine hydrochloride containing an organic solvent. If the amount of the organic solvent (X) is too large, it is preferred to preliminarily remove as much of the organic solvent (X) as possible by an ordinary drying technique (such as vacuum drying).

The paroxetine hydrochloride containing an organic solvent (X) is preferably solid or oily, in particular solid. When the paroxetine hydrochloride containing an organic solvent is solid, the solid may be crystalline or amorphous. The organic solvent (X) may be present in the paroxetine hydrochloride in any state without particular restrictions. For example, when the paroxetine hydrochloride is crystalline, the organic solvent (X) may (or may not) be held in the crystals through salvation, and when the paroxetine hydrochloride is amorphous or oily, the organic solvent (X) may be blended with the paroxetine hydrochloride. Further, the organic solvent (X) may or may not be chemically bonded to paroxetine.

In the present invention, paroxetine hydrochloride containing an organic solvent (X) is neutralized first, preferably with a base capable of forming a salt upon reaction with hydrochloric acid. The base is preferably selected so that the base forms a water soluble salt upon reaction with hydrochloric acid. As the base, inexpensive and readily available bases containing alkali metals are preferred, and particularly preferred are sodium hydroxide, potassium hydroxide, sodium carbonate and potassium carbonate and the like. Sodium hydroxide is extremely particularly preferable because it is available with high purity. It is preferred to use at least equal moles of a base, particularly from 1 to 10 times as many moles of a base, as the paroxetine hydrochloride.

The paroxetine hydrochloride containing an organic solvent (X) preferably gets neutralized in the presence of a medium. The medium may or may not be capable of dissolving the paroxetine hydrochloride. The paroxetine hydrochloride is preferably dissolved or suspended in the medium.

The medium used for the neutralization is preferably a known organic solvent (hereinafter referred to as a first solvent) or water. The first solvent is not particularly restricted and may be the same as or different from the organic solvent (X) in the paroxetine hydrochloride. In particular, when a water soluble base is used for the neutralization, the neutralization is preferably carried out in the presence of water as a medium.

The neutralization is usually conducted by adding a base to paroxetine hydrochloride containing an organic solvent (X). The neutralization temperature may be within the temperature range for ordinary chemical reactions and is preferably from 10 to 35° C. in view of operation, in particular around room temperature (15-30° C.). When the neutralization is accompanied by an evolution of heat of neutralization, the neutralization is preferably carried out with cooling. The neutralization of paroxetine hydrochloride containing an organic solvent (X) gives paroxetine (i.e., paroxetine free base). The free base is usually oily.

In the present invention, a salt of paroxetine with an acid other than hydrochloric acid (hereinafter “an acid other than hydrochloric acid” will be referred to simply as “an acid”) is formed next. In a preliminary step prior to the formation of a salt of paroxetine with an acid, the paroxetine free base is preferably dissolved in an organic solvent (preferably hydrophobic) and then concentrated to remove the organic solvent (X) held in the crystals through salvation. The preliminary step is preferably carried out in accordance with the following specific procedure, procedure 1 or procedure 2.

Procedure 1) When the paroxetine free base is blended with a hydrophilic organic solvent (X) or a hydrophilic first solvent, the free base is dissolved in a hydrophobic organic solvent, and the resulting solution is washed with water or a sodium chloride aqueous solution sufficiently to remove the hydrophilic organic solvent. If part of the hydrophilic organic solvent remains unremoved, the washed solution is concentrated under reduced pressure or ordinary pressure.

For the concentration, another organic solvent capable of forming an azeotrope with the hydrophilic solvent (hereinafter referred to as a second solvent) may be added to remove the hydrophilic organic solvent through formation of an azeotrope. The second solvent is preferably hydrophobic, and the choice of the hydrophobic solvent preferably depends on the type of the hydrophilic solvent.

Procedure 2) When the paroxetine free base is blended with a hydrophobic organic solvent (X) or a hydrophobic first solvent, it is preferred to add another solvent capable of forming an azeotrope with the hydrophobic solvent (hereinafter referred to as a third solvent). The third solvent is preferably hydrophilic. Subsequent concentration under reduced pressure or ordinary pressure allows complete removal of the hydrophobic solvent.

Among the solvents usable as the second or third solvent, preferable examples of hydrophobic solvents are benzene, toluene, xylene, hexane, heptane, octane and the like. The second and third solvents are preferably selected from those which do not form solvates with paroxetine salts other than the hydrochloride. The amount of the second or third solvent is preferably from 1 to 100 times as large, by mass, as that of the organic solvent blended with the paroxetine free base.

In the present invention, then an acid is added to the paroxetine free base. The acid may be any acid, inorganic or organic, other than hydrochloric acid that forms a salt with an amine.

Examples of inorganic acids include bromic acid, iodic acid, hydrofluoric acid, nitric acid, sulfuric acid, sulfurous acid, thiosulfuric acid, phosphoric acid, hydrophosphorous acid, carbonic acid and the like.

Examples of organic acids include formic acid, acetic acid, propionic acid, n-butyric acid, isobutyric acid, nvaleric acid, isovaleric acid, caproic acid, capric acid, lauric acid, myristic acid, palmitic acid, stearic acid, oleic acid, linoleic acid, chloroacetic acid, dichloroacetic acid, trichloroacetic acid, bromoacetic acid, trifluoroacetic acid, oxalic acid, malonic acid, succinic acid, glutaric acid, adipic acid, pyruvic acid, cyanoacetic acid, oxanilic acid, benzoic acid, o-toluic acid, m-toluic acid, p-toluic acid, o-methoxybenzoic acid, m-methoxybenzoic acid, p-methoxybenzoic acid, o-nitrobenzoic acid, m-nitrobenzoic acid, p-nitrobenzoic acid, salicylic acid, m-hydroxybenzoic acid, phydroxybenzoic acid, gallic acid, anthranilic acid, maminobenzoic acid, p-aminobenzoic acid, vanillic acid, nicotinic acid, 2-furanic acid, 2-thiophenic acid, thiophenic acid, 4-quinolinecarboxylic acid, acrylic acid, crotonic acid, methacrylic acid, cinnamic acid, fumaric acid, maleic acid, itaconic acid, atropic acid, propynoic acid, phthalic acid, isophthalic acid, terephthalic acid, homophthalic acid, acridic acid, mandelic acid, tartaric acid, malic acid, lactic acid, methanesulfonic acid, benzenesulfonic acid, p-toluenesulfonic acid, camphorsulfonic acid and the like. Isomers of these acids, if any exist, may be used.

The acid may be a substitution product of such an acid as mentioned above that has a substituent such as an alkyl group, an alkenyl group, alkynyl group, alkoxyl group, a hydroxyl group, an aralkyl group, an aryl group, an acyl group, a carboxyl group, an alkoxycarbonyl group, a carbamoyl group, an amido group, a cyano group, a nitro group, a nitroso group, an amino group, an imino group, an oxime group, an alkoxyimino group, a sulfone group, a sulfonyl group, a sulfinyl group, a mercapto group, an alkylthio group, a phosphonyl group, a phosphinyl group, a silyl group, a halogen atom in place of a hydrogen atom which is not an active hydrogen atom.

The amount of the acid to be used may be the same as or excess over the amount of paroxetine, preferably from 1 to 2 times as many moles, in particular from 1 to 1.1 times as many moles, as that of paroxetine. The temperatures during the formation of a salt with an acid may be within the temperature range for ordinary chemical reactions and is preferably from 15 to 70° C., in particular from 15 to 60° C., in view of advantageous operation. When the formation of a salt with an acid is accompanied by evolution of heat of neutralization, it is preferably carried out with cooling.

The acid is preferably added to a solution of the paroxetine free base. For example, when the paroxetine free base is obtained in solution, an acid may be added directly. In the present invention, in view of the subsequent crystallization, it is preferred to add a solvent (hereinafter referred to as a fourth solvent) which permits adequate crystallization of the paroxetine salt so that the crystals are readily separated by filtration.

As the fourth solvent, a hydrophobic solvent or a mixed solvent comprising a hydrophobic solvent and a hydrophilic solvent is preferably used. The fourth solvent may be the same as or different from the organic solvent (X). Because the crystals are made of a paroxetine salt other than paroxetine hydrochloride, the fourth solvent may be the same as the organic solvent (X), and the fourth solvent is preferably selected so as to be readily removable, even if it remains in the crystals of a paroxetine salt.

The fourth solvent may be a low-boiling solvent or a high-boiling solvent. Use of a low-boiling solvent (preferably having a boiling point of 150° C. or below) as the fourth solvent makes it possible to remove the residual solvent in crystals by vacuum drying. When a high-boiling solvent is used in view of the yield of crystals, the high-boiling solvent can be removed by washing the precipitated crystals with a low-boiling solvent.

The fourth solvent is preferably selected from solvents which hardly remain in the paroxetine salt after crystallization (such as those which do not form solvates with the paroxetine salt) or are readily removable from the crystals (for example, by washing, vacuum drying or other means) even if they remain in the crystals. The fourth solvent may, for example, be an alkane (such as pentane, hexane, heptane, octane or cyclohexane), an aromatic hydrocarbon (such as benzene, toluene or xylene), an ether (such as diethyl ether, tetrahydrofuran or 1,2-dimethoxyethane), a halohydrocarbon (such as dichloromethane, chloroform, carbon tetrachloride, 1,2-dichloroethane, chlorobenzene or dichlorobenzene), an ester (such as methyl acetate or ethyl acetate), a nitrile (such as acetonitrile) or an alcohol (such as methanol, ethanol or 2-propanol). The amount of the solvent is preferably from 1 to 50 times by mass that of paroxetine.

The resulting paroxetine salt is the salt of paroxetine with the above-mentioned acid and is not the hydrochloride. The paroxetine salt usually crystallizes upon standing or stirring. Seed crystals may be added to speed up crystallization, if necessary.

Crystallization is preferably accomplished by adding an acid while stirring a paroxetine solution. If crystallization is so fast as to make stirring difficult, it is prefer to add the acid in a different way. For example, heating the paroxetine solution during the addition of the acid retards the crystallization. The heating temperature is preferably 30-100° C., in particular 30-70° C. The acid is preferably added in at least two portions. It is preferred to adjust the amount of the acid added per unit time in accordance with how the crystallization proceeds. If heating is done, it is preferred to cool the paroxetine solution again to secure a sufficient amount of crystals. The cooling temperature is preferably −10 to 25° C. The crystals can be recovered by ordinary vacuum filtration or centrifugal filtration.

When the paroxetine salt is crystallized out of a high-boiling solvent which is difficult to vaporize, the solvent can remain in the crystals in an amount of 0.1 mass % or above. In such a case, the residual solvent can be removed by washing the crystals with a solvent having a lower boiling point. For example, when a paroxetine salt is crystallized from toluene, the residual toluene is removed by washing the crystals with a solvent having a lower boiling point than toluene (such as hexane) after separation, and then the lower-boiling solvent is readily removed by an ordinary drying method such as vacuum drying.

The crystalline paroxetine salt (which is not the hydrochloride) obtained by the process of the present invention is a paroxetine salt substantially free from an organic solvent. In the present invention, “substantially free from an organic solvent” means that an organic solvent is completely absent or, if any, present in the paroxetine salt in an amount of 0.1 mass % or below. Herein, the organic solvent includes not only the organic solvent (X), but also all the organic solvents usable for the process of the present invention.

The paroxetine salt substantially free from an organic solvent obtained by the process of the present invention may also be a paroxetine salt substantially free from water (i.e., may not contain more than 0.1 mass % of water). Namely, because it is possible that substantially all the water has been removed from the paroxetine salt obtained by the process of the present invention, various troubles caused by water can be avoided. In particular, it is preferred that the paroxetine salt substantially free from an organic solvent is paroxetine acetate, because of the advantage that it has very little hygroscopicity and stays substantially free from both an organic solvent and water for a long time even during a long storage. Crystalline paroxetine acetate substantially free from an organic solvent (preferably free from water, too) is novel.

The paroxetine salt substantially free from an organic solvent obtained by the process of the present invention can be converted to the hydrochloride, if necessary, by treatment with hydrogen chloride. Therefore, according to the process, the hydrochloride can be obtained in a state substantially free from organic solvents and water. The hydrochloride in such a state can not be obtained just by dissolving paroxetine hydrochloride containing an organic solvent in an organic solvent again and recrystallizing it. Namely, the paroxetine hydrochloride obtained by recrystallization usually contains an organic solvent.

Paroxetine salts other than the hydrochloride obtained by the process of the present invention are useful as medicines, like paroxetine hydrochloride, (WO99/40084, WO00/01692, WO00/08016 and European J. Pharmacol., 1987,47,351). Therefore, these paroxetine salts can be used as medicines without converting them into the hydrochloride.

Furthermore, the paroxetine salts and paroxetine hydrochloride obtained by the process of the present invention may be converted to amorphous paroxetine salts or amorphous paroxetine hydrochloride by spray-drying their solutions, if necessary, and secondary drying (such as vacuum drying) for desired uses.

The paroxetine salts and paroxetine hydrochloride substantially free from organic solvents obtained according to the present invention are useful as antidepressants.

### EXAMPLES

Now, the present invention will be described in further details by reference to specific Examples, but the present invention is by no means restricted thereto. Hereinafter, liter is represented as L. The organic solvent content and the water content were determined by gas chromatography and the Karl Fischer method, respectively.

### Example 1

Solid paroxetine hydrochloride (200 g) containing 2.7 mass % of 2-propanol was added to toluene (930 mL) and water (1.33 L), and aqueous potassium hydroxide (prepared by dissolving 182 g of potassium hydroxide in 590 mL of water) was added with stirring. The resulting solution was stirred for 50 minutes and then allowed to stand. After separation of the toluene layer, the remaining aqueous layer was extracted with toluene (930 mL), and the toluene extract was combined with the previously separated toluene layer. Then, the toluene layer was washed with 14 mass % aqueous sodium chloride (690 g), dried over magnesium sulfate and concentrated to 0.2 L to give paroxetine free base.

To the paroxetine free base, toluene (0.9 L) was added, and then acetic acid (32.85 g) was added under cooling with ice-cold water. Then, the solution was heated on an oil bath at 70° C. for 2 hours and allowed to cool for one day for crystallization. The precipitated crystals were collected by vacuum filtration, washed with hexane (0.2 L), stirred in hexane (0.49 L) for 1 hour, collected by vacuum filtration and dried by vacuum drying (230 Pa (absolute pressure), 25° C.) to give crystalline paroxetine acetate (189.8 g, yield 89%).

An analysis of the remaining organic solvents in the paroxetine acetate thus obtained revealed absolute absence of 2-propanol. The total content of toluene and hexane was below 0.1 mass %. The water content was below 0.1 mass %.

### Example 2

To the paroxetine acetate obtained in Example 1 (189.8 g), dehydrated ethanol (0.89 L) was added in a nitrogen atmosphere, and an ethanol solution (0.25 L) containing 41.6 g of hydrogen chloride was added. The resulting solution was stirred for 30 minutes at 40° C. The resulting paroxetine hydrochloride solution was filtered and concentrated, and when the absence of acetic acid was confirmed, the residue was dissolved in dehydrated ethanol (0.58 L). The ethanol solution was spray-dried through a nitrogen-circulating spray drier for organic solvents (GS-310, manufactured by Yamato Scientific Corporation) and then vacuum-dried (170 Pa (absolute pressure), 25° C., 68 hours) to give powdery amorphous paroxetine hydrochloride (115.3 g).

### Example 3

The same procedure as in Example 1 was followed until the concentration of the toluene layer, and the resulting paroxetine free base (93 g) was put in toluene to give a toluene solution (500 mL). It was heated to 55° C., and acetic acid (4.1 g) was added. Several grains of crystalline paroxetine hydrochloride were added with stirring, and after crystallization started, acetic acid (12.3 g) was added dropwise over 30 minutes at 55-68° C. with heating. The solution was stirred at 68° C. for 1.5 hours and then allowed to cool at 25° C., and the precipitated crystals were collected by vacuum filtration, washed with hexane (90 mL), then stirred in hexane (240 mL) at 25° C. for 1 hour and collected by vacuum filtration. The crystals were vacuum-dried under the same conditions as in Example 1 to give crystalline paroxetine acetate (94.4 g, yield 89%). The paroxetine acetate thus obtained did not contain 2-propanol at all. The total content of toluene and hexane was below 0.1 mass %. The water content was below 0.1 mass %.

### Example 4

Solid paroxetine hydrochloride (500 g) containing 2.5 mass % of 2-propanol was put in toluene (1200 mL) and water (660 mL), and aqueous sodium hydroxide (prepared by dissolving 86 g of sodium hydroxide in 260 mL of water) was added with stirring. The resulting solution was stirred for 60 minutes for 30° C. and allowed to stand. The toluene layer was separated, washed with 14 mass % aqueous sodium chloride (245 g) and concentrated to give paroxetine free base.

A solution (2.6 kg) of 450 g of the paroxetine free base in toluene was heated to 30° C., and acetic acid (27° C.) was added. After crystallization started, the temperature was raised to 60° C., and acetic acid (54 g) was added dropwise at 60-70° C. After 0.5 hour of stirring at 70° C., the solution was cooled to 50° C., and the precipitated crystals were collected by filtration. The collected crystals were washed with toluene (500 mL) and vacuum-dried (130 Pa (absolute pressure), 40° C.) to give crystalline paroxetine acetate (495 g, yield 93%). The crystalline paroxetine acetate contained 2-propanol in an amount of less than 0.1 mass %. The toluene content was below 0.1 mass %. The water content was below 0.1 mass %.

### Example 5

To the paroxetine acetate (130 g) obtained in Example 4, dehydrated ethanol (0.4 L) was added, and an ethanol solution (0.2 L) containing 13 g of hydrogen chloride was added. After 2 hours of stirring at 40° C., dehydrated ethanol (1.3 L) was added intermittently, and the resulting solution was vacuum-dried to 0.7 L. The concentrated paroxetine hydrochloride solution was filtered.

The resulting paroxetine hydrochloride solution in ethanol was spray-dried through a nitrogen-circulating spray drier for organic solvents (GS-310, manufactured by Yamato Scientific Corporation) at a feed rate of 0.6 L/hour at the circulating gas rate inlet temperature of 90° C. and then vacuum-dried (40° C., 130 Pa (absolute pressure)) to give anhydrous amorphous paroxetine hydrochloride (95 g).

The 2-propanol content, toluene content and water content in the resulting amorphous paroxetine hydrochloride were below 0.1 mass %, respectively. The ethanol content was less than 1.1%.

### INDUSTRIAL APPLICABILITY

According to the present invention, it is possible to obtain paroxetine salts substantially free from organic solvents by removing organic solvents from paroxetine hydrochloride under quite mild conditions without using special reagents or operations. The process of the present invention makes it possible to remove organic solvents in a short step without means such as heating, prevent decrease in yield resulting from thermal decomposition of paroxetine and recover paroxetine salts in high yields. The process of the present invention which can give paroxetine salts substantially free from water makes it possible to provide paroxetine salts useful as ingredients of medicines with no troubles caused by water. The resulting paroxetine salts may, if necessary, be converted to the hydrochloride or an arbitrary salt by salt exchange or other means. The paroxetine hydrochloride and paroxetine salts other than the hydrochloride obtained by the process of the present invention can be converted to an amorphous form substantially free from organic solvents by spray drying or other means.

The entire disclosure of Japanese Patent Application No. 2000-280037 filed on Sep. 14, 2000 including specification, claims and summary is incorporated herein by reference in its entirety.

What is claimed is:

1. A process for producing a paroxetine salt substantially free from an organic solvent, which comprises neutralizing paroxetine hydrochloride containing an organic solvent into paroxetine, then forming a salt of paroxetine with an acid other than hydrochloric acid, and crystallizing the salt from an organic solvent to give a salt other than paroxetine hydrochloride.

2. The process according to claim 1, wherein the acid other than hydrochloric acid is acetic acid.

3. The process according to claim 1, wherein the paroxetine hydrochloride containing an organic solvent is paroxetine hydrochloride containing 2-propanol.

4. The process according to claim 1, wherein the paroxetine obtained by neutralized paroxetine hydrochloride containing an organic acid is dissolved in a hydrophobic solvent, then concentrated and allowed to form a salt with an acid other than hydrochloric acid.

5. The process according to claim 1, wherein crystallization is carried out in an organic solvent which hardly remain in a paroxetine salt or is readily removable from the crystals even if it remains in the crystals.

6. The process according to claim 1, wherein the organic solvent is an organic solvent which does not form a solvate with the paroxetine salt.

7. The process according to claim 1, wherein from 1 to 2 times as many moles of an acid other than hydrochloric acid as paroxetine is added to paroxetine to form a salt of paroxetine with the acid other than hydrochloric acid.

8. The process according to claim 1, wherein the paroxetine salt substantially free from an organic solvent is substantially free from water.

9. A process for producing paroxetine hydrochloride substantially free from an organic solvent, which comprises treating the paroxetine salt substantially free from an organic solvent obtained by the process according to claim 1 with hydrogen chloride.

10. A process for producing an amorphous paroxetine salt, which comprises spray drying the paroxetine salt substantially free from an organic solvent obtained by the process according to claim 1 in solution and secondary drying.

11. A process for producing an amorphous paroxetine hydrochloride, which comprises spray drying the paroxetine hydrochloride substantially free from an organic solvent obtained by the process according to claim 9 in solution and secondary drying.

12. Crystalline paroxetine acetate substantially free from an organic solvent.
